(19) United States
(12) Patent Application Publication
(10) Pub. No.: US 2007/0265507 A1
(43) Pub. Date: Nov. 15, 2007

(54) VISUAL ATTENTION AND EMOTIONAL RESPONSE DETECTION AND DISPLAY SYSTEM

(75) Inventor: Jakob de Lemos, Copenhagen (DK)

Correspondence Address: PILLSBURY WINTHROP SHAW PITTMAN, LLP;Eric S. Cherry - Docketing Supervisor, P.O. BOX 10500, MCLEAN, VA, 22102, US

(73) Assignee: iMotions Emotion Technology ApS, Copenhagen (DK)

(21) Appl. No.: 11/685,552

(22) Filed: Mar. 13, 2007

Related U.S. Application Data
(60) Provisional application No. 60/781,321, filed on Mar. 13, 2006.

(51) Int. Cl. A61B 5/00

(52) U.S. Cl. 600/300

(57) ABSTRACT

The invention is a system and method for determining visual attention, and supports the eye tracking measurements with other physiological signal measurements like emotions. The system and method of the invention is capable of registering stimulus related emotions from eye-tracking data. An eye tracking device of the system and other sensors collect eye properties and/or other physiological properties which allows a subject's emotional and visual attention to be observed and analyzed in relation to stimuli.

### RELATED APPLICATION DATA

This application claims the benefit of U.S. Provisional Application No. 60/781,321 filed on Mar. 13, 2006. The entire teachings of the above application are incorporated herein by reference.

### FIELD OF INVENTION

The invention relates to computer-implemented systems and methods for determining and displaying visual attention and other physiological signal measurements (e.g., emotional response information of a person in response to presented stimuli) by collecting and analyzing eye movement, other eye properties and/or other data.

### BACKGROUND OF INVENTION

Eye tracking systems in general are known. Emotional response detection systems in general are known. However, various limitations and drawbacks exist with these known systems.

The display of visual attention data in general is known. However, various limitations and drawbacks exist with known systems. Additionally, the simultaneous display of visual attention data and corresponding emotional response has not traditionally been used.

Other drawbacks and limitations exist with known systems.

### SUMMARY OF INVENTION

One aspect of the invention relates to a system and method of determining and displaying visual attention information and emotional response information related to stimuli presented to a subject (e.g. a person being tested). According to one aspect of the invention, visual attention information (for example, fixation points and saccades) is determined and then displayed, for example, using a gaze plot with a spotlight feature. A fixation point may be a point or area of a stimulus (e.g., visual image) on which a subject focused for at least a minimum amount of time. As used herein, a fixation point may also refer to a fixation area identified by multiple fixation points and saccades. A spotlight may be an aggregation of fixation points visualized through an aggregated transparency on a black mask (or other type of mask) layered above the stimulus. For example, based on selectable thresholds (e.g., administrative user selected thresholds) and/or other parameters, the spotlight feature may be used to indicate one or more fixation points. Aggregated fixation points may also be used with temporal ordering to create attention points. Attention points may be visualized through numbering to indicate the temporal ordering of the aggregation of fixation points (e.g., spotlight). If desired, other points or areas (e.g., ones that do not meet the threshold or other parameters) may be selectively distinguished from the fixation points.

One advantage of this is that the gaze plot with spotlight feature can graphically depict what portions of a stimulus that a subject fixated upon (and, if desired, obscure areas that the subject did not fixate on). This enables an interested party (e.g., a marketing consultant or other entity) to easily see what portions of the stimuli the subject fixated on and/or which portions were not fixated on for a given stimuli. While this information alone is useful, by itself it does not indicate whether the subject had an emotional response to the stimuli as a whole, much less an emotional response associated with one or more given fixation points. Nor does it indicate, if there was an emotional response, the type of emotion (e.g., a positive emotion or a negative emotion) or how strong the emotion was.

According to another aspect of the invention, a subject's emotional response can be determined and displayed for a given stimuli and/or for fixation points of a stimuli. A fixation point that is determined to correspond to an emotional response may be referred to as an interest point. Emotional response information (e.g., type and/or strength of emotion) may be displayed simultaneously with visual attention information (e.g., displaying emotional response information simultaneously with a gaze plot or other display of visual attention information). Interest points may also be displayed alone or simultaneously with visual attention and/or emotional response information.

The displayed emotional response information may include display of one or more of emotional valence and/or emotional arousal. This information can indicate the type of emotion (e.g. a positive one or a negative one) and/or the strength of the emotion. For interest points, the type and strength of the emotional response (among other things) can be determined and/or displayed. The display may use different display characteristics to distinguish between different fixation points, attention points, temporal ordering, interest points and/or emotion types and strengths.

Emotional response information can be determined in any of a number of ways. Various emotion detection techniques are known (e.g., reading facial movement, galvanic skin response and various other techniques). According to one synergistic embodiment of the invention, the emotional response information can be detected based, at least in part, on the subject's eye properties (e.g., eye movement, blink rate, pupil dilation and/or other eye properties). Advantageously, this enables (if desired) the same eye tracking device that is used to collect visual attention data to collect emotional response data. Various other emotion detection techniques may used with, or instead of, eye property detection.

Various configurations, features and functions may be used in various combinations within the scope of the invention. By way of example only, and without limitation, only some examples are described herein. According to one embodiment, a system may include, among other things, a set-up module (e.g., for enabling set-up of one or more of test parameters, subject profile, stimuli parameters, calibrations and/or other set-up parameters), a stimuli presentation module (e.g., for managing the storage and presentation of stimuli), a data collection module, an analysis module (e.g., for analyzing the collected data to determine visual attention and/or emotional response) and an output module for selectively outputting information, including information relating to the determined visual attention and/or emotional response information, among other things. The output may be in any of a number of different forms, can include various types of information and can include various levels of detail.

According to one aspect of the invention, the output module enables the output of visual attention information, such as a gaze plot with a spotlight feature and/or attention points (e.g., as explained above). According to another aspect of the invention, the output module enables output of a subject's emotional response information and/or interest point in motion. Other types and combinations of outputs may be selected.

Any of the outputs can be for: a single stimulus presented to a single subject (e.g., a person); an aggregate output for a number of stimuli presented to the same subject; an aggregate output of a single stimulus presented to a group of subjects; and/or a number of stimuli presented to a group of subjects. Any of the outputs can include a “snapshot” view (e.g., a single result for information determined by sampling over a specific period of time) and/or a time series display (e.g. a series of snapshots over time), animation, and/or a video (e.g., a relatively continuous, motion display showing the subject's eye movement and/or other information over a period of time). According to this aspect of the invention, the visual attention information and/or emotional response information may be recorded and played back to demonstrate the subject's visual attention and/or emotional response in a video replay mode. Playback controls may be provided.

In one embodiment, the system and method of the invention may be configured to determine the visual attention of a subject regarding one or more specified stimulus and/or various portions (e.g., selected areas) of the stimuli.

After any necessary and/or desired set-up (e.g., collection of background variables including subject's age, address, gender, and/or other demographic information) and/or calibration steps are performed, visual attention information (e.g., fixation and/or saccades with respect to a visual stimulus presented on a computer display) may be determined, at least in part, by tracking eye properties (including, for example, collecting data relating to eye position, eye movement, rate of eye movement, and/or other eye properties). The visual attention information that is determined may include fixation points (gaze) and saccades (e.g., the path between fixation points) and/or other information. According to one aspect of the invention, this enables a subject's eye movements, which may have previously been calibrated to display device coordinates, to be correlated to a visual stimulus or portions thereof. In general, the visual attention information relates to what portion(s) of the stimulus the subject is looking at one or more points in time. All or some points/areas of the stimulus at which the subject looked may be identified and displayed or only points/areas meeting certain criteria may be displayed. For example, threshold values may be set to display only points/areas at which a subject fixated on for at least a predetermined minimum period of time or points/areas to which the subject came back to a number of times. Other criteria may include temporal ordering of the points/areas of the stimulus that are identified as fixations. From a business perspective, a service provider may use the software/system to run test centers that subjects visit. In this scenario, one or more test leaders (and/or administrative users) may be used to assist/guide the subjects in conjunction with the testing. Self-operated and/or semi-automated test centers (e.g., kiosks, PC, etc.) may also be used with or without a test leader. Remotely supervised testing may also be implemented

The service provider may collect fees on a variety of bases including, but not limited to, a per test fee, a per stimuli fee, per subject, per segment of population, and/or other bases. Additionally, the amount of fee may vary depending on the type and/or detail of the output. For example, a simple output (e.g., gaze plot only) may be provided for a first fee. A gaze plot with the spotlight feature may be a second fee. A simultaneous display of a gaze plot with basic emotional response information may be a third fee. Adding more detailed emotional response information may be a fourth fee. Other business models for such service providers may be implemented.

According to another business method, a service provider may operate a remotely accessible (via the Internet or other network) test facility with which subjects can interact remotely therefrom. The subject can access the remotely accessible test facility in any of a number of ways, including but not limited to, via a test center, a kiosk, a home or work computer, a mobile wireless device or otherwise. Fees may be charged as indicated above or otherwise.

According to another business model, the software may be licensed. As detailed below, the licensing may be on a modular basis. For example, the visual attention module and/or emotional response module may respectively include a core visual response engine and a core emotional response engine. The core engines may each be licensed for a base fee. Separate plug-ins (or other modules) to provide enhanced functionality and/or greater level of detail may be provided for separate fees. Yet another business model may require a predetermined type of device to be licensed with the software. For example, a serial number of the eye tracking device may be determined to be an acceptable device before it is allowed access to software functions. Other licensing models can be used. An invoice module may monitor system activities to facilitate in any invoicing that may be necessary or desired.

To accommodate these and other business methods, any of the set-up/calibration and/or running of tests may be done manually, automatically and/or semi-automatically. If desired, real-time monitoring of the results may be made available locally or remotely.

Various other features and functions may be used with one or more aspects of the invention. Not all of the features and function described herein need to be used in all cases. Any combination of features and/or functions may be used as desired. The examples provided below are for ease of understanding. The invention is not limited to any specific implementation.

### BRIEF DESCRIPTION OF THE DRAWINGS

FIG. 1 is an example of a high level representation of a method according to one embodiment of the invention.

FIG. 2 schematically illustrates a functional block diagram of an example of portions of a system for determining visual attention and emotional response information relating to a stimuli presented to a subject according to an embodiment of the invention.

FIG. 3 is an illustration of an exemplary functional block diagram of portions of a system according to one embodiment of the invention.

FIG. 4 is a high-level exemplary flow diagram of methods for setting up and running tests and analyzing test results according to various embodiments of the invention.

FIG. 5 is an illustration of an exemplary visual stimulus, according to an embodiment of the invention.

FIG. 6 is an illustration of one example of a output generated by the system, according to an embodiment of the invention.

FIG. 7 depicts examples of some components of outputs according to some aspects of the invention.

FIG. 8 is an illustration of an output generated by the system, according to an embodiment of the invention.

### DETAILED DESCRIPTION

The systems and methods of the invention have a broad range of applicability. For purposes of clarity, one scenario in which these features are beneficial will be described. By way of example, one scenario relates to situations where a subject (e.g., an individual) is tested by presenting stimuli and/or survey questions to the subject (e.g., to determine the subjects reaction to advertisements, a new product, a new feature of a product and/or packaging for a product, among other things). For convenience, the invention will be discussed primarily in the context of such testing. This is not intended to limit the invention thereto. The invention can be used in a wide variety of other scenarios and applications as well.

As used herein, “testing” and/or “study/survey” may broadly refer to a wide variety of activities (e.g., advertising or marketing studies or surveys for new products, new features, new packaging or other testing or studies). A “subject” may, for example, include a person, animal or other test subject being tested. Stimuli may include any type of sensory stimuli corresponding to any one or more of the five senses (sight, sound, smell, touch, taste) and/or other stimuli. Visual stimuli may be presented on a display (e.g., as a single image, two or images sequentially or simultaneously, as a video, or otherwise).

Examples of visual stimuli, for instance, may include pictures, artwork, charts, graphs, text, movies, multimedia presentations, interactive content (e.g., video games), or other visual stimuli. Stimuli may be recorded (on any type of media) and/or include live scenarios (e.g., driving or riding in a vehicle, etc.) Various stimuli and/or stimuli types may be combined. For any test or other scenario, stimuli may be selected based on the purpose and need. For example, in an advertising context, stimuli may correspond to a product advertisement to determine the overall reaction to the stimuli (the ad) and more detailed information (e.g., where the subject's attention is drawn to on the ad, and what emotions are felt while perceiving the stimuli or portion thereof).

A used herein, an “administrator,” or “administrative user” (if one is used) may refer to the person that performs at least some of the setup operations related to a test (and/or other functions). For example, an administrator may interact with the system to input test setup critical parameters including for example, stimuli parameters, subject participants, background variables (e.g., age, gender, location, etc.) and/or parameters.

A study/survey leader, (if one is used) may assist in running the actual test. The administrator and the leader may be the same person or different people.

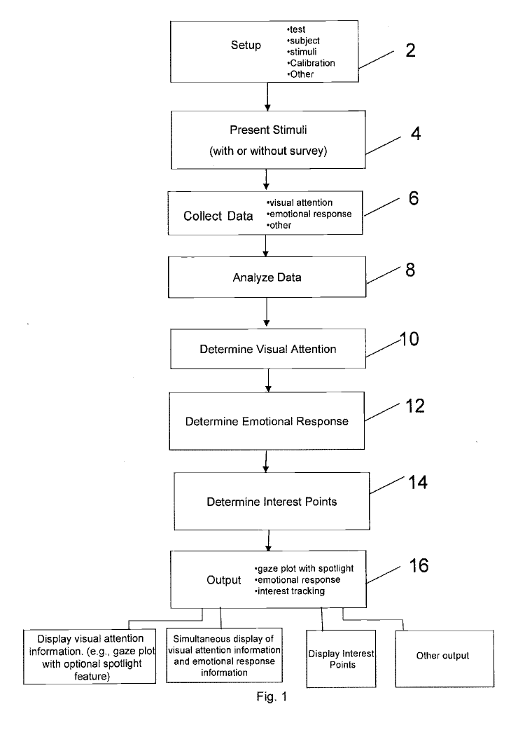

FIG. 1 illustrates an example of a high level diagram of a method according to one embodiment of the invention. Various set-up/calibration steps may be performed (Step 2). Set-up and calibration techniques, in general, are known. Examples of these steps may include, among other things, test set-up, subject setup, stimuli setup, various calibration steps and/or other steps. According to one novel aspect of the invention, segmentation setup may include collecting both independent and dependent background variables. Stimuli may be presented to a subject (Step 4). If desired, survey questions may also be presented to the subject. Survey presentation and survey results collection, in general, are known. However, according to one novel aspect of the invention, survey responses, visual attention information and emotional response information may be correlated.

Data relating to the subject's reactions to the stimuli (including visual attention data and/or emotional response data) are collected (Step 6). During and/or after stimuli presentation, the collected data (and/or other desired information) may be analyzed (Step 8). The analysis may include determining visual attention information (Step 10), emotional response information (Step 12), interest point(s) (Step 14) and/or other information (e.g., physiological information associated with a subject with respect to one or more presented stimuli). Analysis data may then be stored and/or selectively output (Step 16). The output can be in any of a variety of forms, including a computer displayed report or other type of output. One aspect of the invention relates to specific types of output as detailed below.

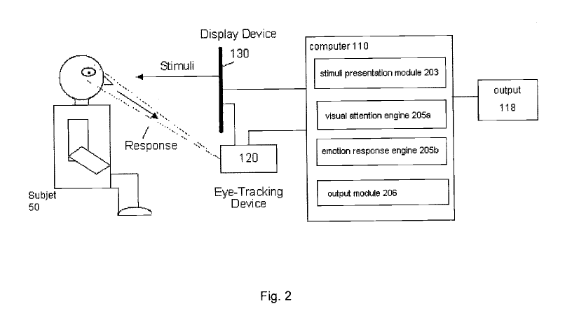

FIG. 2 illustrates one example of parts of a simplified view of a system that can be used to implement some aspects of the invention. As illustrated, the system may include at least one or more of an eye tracking device 120, a display device 130, and computer device 110. The Computer 110 may be programmed (or access a computer/server that is programmed) with at least one or more of a stimuli presentation module 203, a visual attention engine 205a, and/or emotional response engine 205b. An output module 206 may be used to generate output 118. One or more storage devices (not shown in FIG. 2 for simplicity) may store stimuli, data, analysis results and/or other information.

In operation, a subject 50 may be positioned in proximity to display device 130. Stimuli presentation module 203 may cause selected stimuli to be displayed on the display device 130 to expose subject 50 to one or more visual (or other) stimuli (e.g. stimuli displayed on display device 130 and/or other device). One or more data collection devices (e.g., eye tracking device 120 and/or other data collection devices) may collect data and/or record information regarding the subject's responses. The collected data may include a desired number of discrete samples (e.g., 50-60 samples per second or any other desired frequency) over a predetermined period or variable period of time (e.g., 1-3 seconds or any other period). Alternatively or in addition, the collected data may include a continuous sampling (e.g. a video) for a fixed or variable period of time. The collected data may include eye movement and other eye properties, physiological data, environmental data and/or other data relating to the subject's response to various stimuli. Manual input from the user may also be received.

According to one advantageous aspect of the invention, the eye tracking device 120 may be integrated with and/or mounted on or in the display device 130. However, these devices may also be implemented as separate units based on various detection environments and scenarios. A display device 130 may include a monitor, touch screen, LCD screen, and/or other display devices. If desired, a simple USB type video camera may be used as the eye-tracking device 120. This (or other eye-tracking devices) may be integrated with or mounted to any usable display. One example of an integrated eye-tracking and display device is the Tobii 1750 Eye-tracker, commercially available from Tobii Technology AB.

The eye-tracking device may include or interact with a software program to control the eye-tracker and collection of data thereby. For example, the eye-tracking device may include Clearview™ software (provided by Tobii). Other eye-tracking software can be used. This software may be a standalone application or maybe bundled with or part of one or more of the other software modules described herein. The eye-tracking software may incorporate one or more of the other software modules. Other eye-tracking devices, displays and/or technology may be used in place of, or with, the various components described herein.

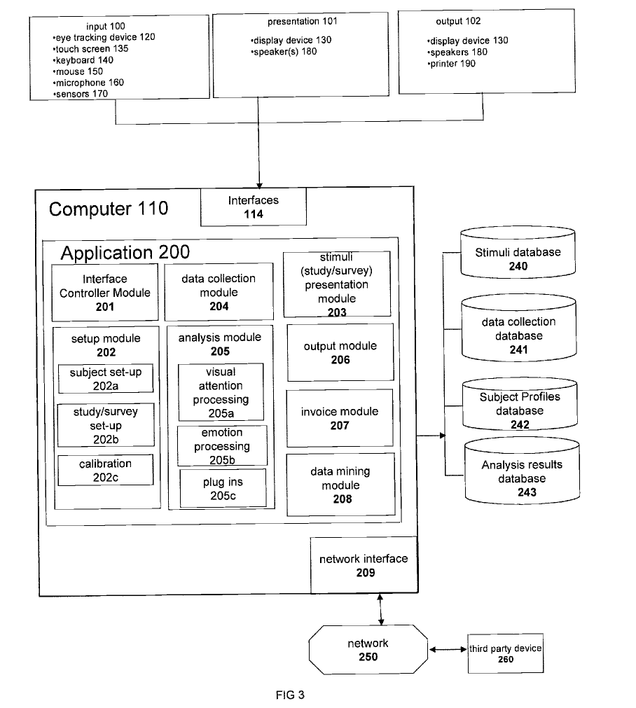

FIG. 3 illustrates a more detailed functional block diagram of a system (and other features), according to one embodiment of the invention. FIG. 3 illustrates a computer 110 having one or more interfaces 114 for interfacing with one or more input devices 100, one or more presentation devices 101 and/or one or more output devices 102. Computer 110 may further be in communication with one or more storage devices, such as stimuli database 240, data collection database 241, subject profiles database 242, analysis results database 243 and/or other storage devices. One or more of databases 240, 241, 242 and 243 may be provided to store stimuli information, collected data, subject profile information, analysis results and/or other data. These databases may be separate databases, as shown for clarity, or one or more may be combined into a single database for storing application system data.

The input devices 100 (e.g., one or more of an eye tracking device 120, touch screen 135, keyboard 140, mouse 150, microphone 160, sensors 170, and/or other input devices) may be used for receiving input (e.g., from a subject 50 or other input). The input may include but is not limited to, information regarding a subject's visual attention, emotional response and/or other responses to stimuli. Other input may include user information received during a set-up/calibration procedure, survey responses and/or other user input, and other desired input. Sensors, such as scent sensors, tactile sensors, sound sensors and/or other sensors may also be used as input devices.

The presentation devices may include, for example, one or more of display device 130, speaker(s) 180, and other presentation devices. Display device 130 may be used for visually displaying and presenting visual stimuli to a subject.

The output devices 102 may include, for example, one or more of a display device 130 (or other display), speakers 180, printer 190, and or other output devices. The display device 130 may include a video display for displaying a video playback of the collected data or a processed version of the collected data. Computer 110 is programmed with, or is in communication with a computer (e.g., a remote server) that is programmed with, a software application (e.g., application 200 illustrated in FIG. 3) to perform the functions described herein. Computer 110 may be a single computer or multiple computers. One or more computers 110 may be located locally (in proximity to the test subject 50) and one or more may be located remotely from the test subject 50 (e.g. at a central test facility) to enable remote testing of subjects and/or remote monitoring of tests. One or more computers 110 can be standalone computers running an application 200. One or more computers 110 can be networked (e.g., via network interface 209 to one another and/or any third party device 260, to enable networked communication there between. This may enable, among other things, browser-based access from one computer to a central computer 110 running application 200. The computer 110 may access the application 200 over a network 250 (e.g., the Internet, an intranet, WAN, LAN, etc.) via any wired and/or wireless communications links.

Application 200 may include one or more computer software programs and/or modules that, among other things, perform functions set forth herein. For example, application 200 may perform functions including one or more of setup/calibration, testing, stimuli presentation, data collection, analysis, output, generation, and/or formatting, invoicing, data mining, among others.

For convenience various ones of the functions may be carried out by various modules 201-209, as shown for example in FIG. 3. One or more modules may be combined and any module shown as a single module may include two or more modules. By way of example, the modules may include at least one or more of an interface controller module 201, a setup module 202, a stimuli presentation module 203a data collection module 204, an analysis module 205, an output module 206, an invoice module 207, a data mining module 208 and/or other modules. Not all modules need to be used in all situations.

One or more interface controller module 201 may be associated with and/or in communication with one or more input devices 100, presentation devices 101, and output devices 102 in any known manner. One or more controllers 201 may be implemented as a hardware (and/or software) component of the computer 110 and used to enable communication with the devices attached to the computer 110. The communication can be conducted over any type of wired or wireless communication link. Secure communication protocols can be used where desired.

Setup module 202 includes sub-modules for one or more of subject setup 202a, stimuli setup 202b, calibration 202c and/or other setup/calibration procedures. These procedures may include those referred to in connection with Step 2 of FIG. 1, among others. Data received by the system during setup/calibration (e.g., background variables, test parameters, stimuli parameters, subject parameters, etc.) may be stored in one of stimuli database 240, subject profile database 242, and/or other databases.

Stimuli presentation module 203 may be provided to facilitate the presentation of stimuli according to stimuli setup information, stored stimuli and/or other stimuli presentation properties. The stimuli presentation module 203 may include or interact with a graphical user interface (not shown) to enable stimuli to be managed (e.g., stored, deleted, modified, uploaded/downloaded, or otherwise managed) by an administrative user or otherwise. Additionally, a user interface can enable one more stimuli and stimuli presentation properties to be selected for use with a particular test or other application.

The data collection module 204 may collect data (e.g., from one or more of input devices 100 or other input devices) during stimuli presentation (and at other times). The data collection module 204 may cause the collected data to be stored in data collection database 241 or other database for later (or real-time) analysis.

Analysis may be done by the analysis module 205 and/or other processor. Analysis module 205 may include sub-modules for visual attention processing 205a, emotional response processing 205b, and/or other sub-modules. If desired, various plug-ins 205c, may be used to enhance the functionality of a core emotional response engine and/or visual attention engine.

Analysis results may be stored in analysis database 243 or other database. The analysis module 205 may process the collected data using one or more error detection and correction (data cleansing) techniques. As such, the collected data may be refined and filtered to decrease signaling noise and other errors. The clean data may be more easily and/or accurately analyzed.

Various plug-ins 205c may be used to offer greater level of detail to and/or additional functions regarding the visual attention and/or emotional response processing. For example, interest points may be determined from the collected data. Some details may include detailed interest points, emotional valence determination, emotional arousal determination, emotion name and type.

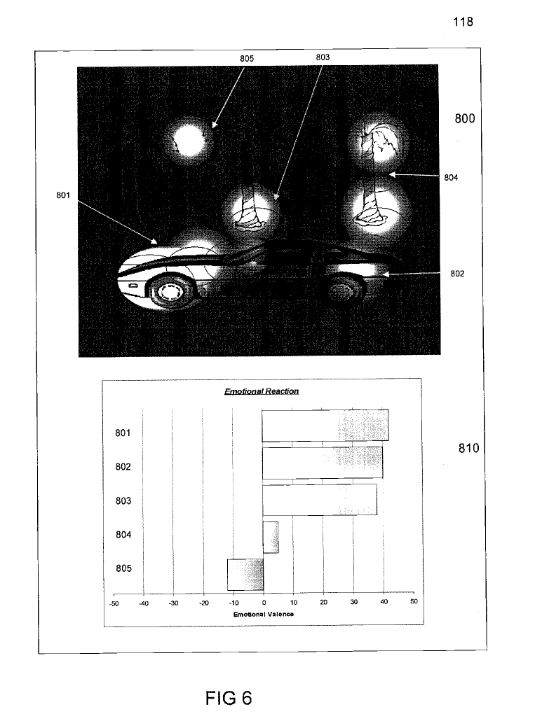

An output module 206 may selectively enable various types of outputs to output from the application 200 to one or more output devices 102. For example, the output module 206 may be used to produce reports based on analysis results. For example, visual attention information and emotional response information may be output and presented with respect to the actual stimuli in the report output 118. Various electronic and/or printed output types may include, but are not limited to, representation in the form of graphs, text, illustrations, gaze plots, emotion meters, audio, and/or video play back, to name a few. Further details and examples of output are set forth in connection with FIGS. 6-8. Other output types and formats may be used

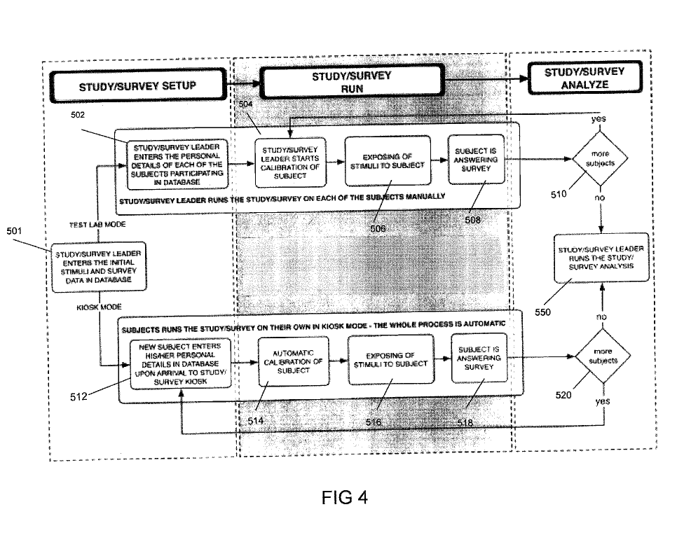

FIG. 4 illustrates examples of methods for carrying out various aspects of one embodiment of the invention. FIG. 4 illustrates a Study/Survey setup phase, a Study/Survey Run phase and a Study/Survey Analysis phase. These phases and/or other phases may be carried out at a test facility (or outside the test facility) in any of a number of ways, including but not limited to, via a test center, a kiosk, a home or work computer, a mobile wireless device or otherwise. Testing may be supervised, semi-supervised or unsupervised. At a test facility the testing may be run by a study/survey leader on each subject manually. Outside of the testing facility, the subject 50 may run the study/survey with or without a study/survey leader. Without a study/survey leader, the subject's emotional state may remain unaltered and unaffected by the presence of a study/survey leader. Alternatively, a combination of aspects from the testing facility and from outside the testing facility may be used during the phases illustrated in FIG. 4. Other testing environments may also be included within the scope of the invention.

In some or all testing environments, there may be Study/Survey setup phase. In this phase the administrator (or other individual) enters or selects the stimuli and/or survey data and other setup parameters (e.g., background variables). This information may be stored in stimuli database 240 and/or other database(s) (step 501).

The stimuli to be presented during the study/survey may be selected using the study/survey setup sub-module 202b of the setup module 202. The selected stimuli may be loaded on the computer 110 and/or stored in stimuli database 240 or other database. Various stimuli sources may be used. Remote stimuli (not shown) may be accessed via network interface 209 over the network 250 (e.g., internet, intranet, etc.) to download stimuli from the remote source such as an advertisement database. Another stimuli source may be a stimuli creation application which may allow the creation and/or customization of stimuli. The creation application may enable multimedia stimuli creation.

Other stimuli presentation properties may also be selected. For example for a given test/study, one or more of the stimuli duration for one or more stimuli, the order of presentation of stimuli (e.g., random presentation of stimuli), whether any stimuli should be simultaneously presented, an/or other stimuli properties may be selected. The parameters for identifying a fixation point may be provided during a set up of stimuli properties (or at other times). For example, this may be based at least on threshold values for dwell time or other parameters. The visual display of spotlight(s) may be set-up to be based on a number of aggregated fixation points or other factors. The attention points may be set-up to visually indicate the temporal ordering (e.g., semitransparent number indicator) of aggregated fixation points with respect to identified spotlights. Interest points may be identified based on fixation point (e.g., as determined by selected criteria) and emotional response (as defined by selected criteria) at the fixation point. For example, it may be specified that if a particular type and/or strength of emotional response is associated with one or more fixation point(s), this may identify an interest point(s). These aspects are discussed in more detail below.

Output presentation properties may also be specified using the setup module 202. The output presentation properties may identify what analysis will be done, output type and/or format, who should receive the output and/or how the output will be received, among other things. For example, the level of information to be included in an output report may be specified using, for example, a presentation format including predetermined templates. The parties to receive the output information and the associated transmission means may also be specified as part of the output presentation properties. For example, the output(s) may be sent to a specified user/device using a predetermined transmission means (e.g., email, phone, FTP, etc.). The output presentation properties may be entered by one or more of the administrator, leader, and subject and/or other individual.

The method of FIG. 4 may also include receiving profile (and/or other) information regarding the subject (e.g., background variables including age, gender, location, etc.) At a testing facility, the leader may enter or guide the subject(s) to enter details of the participating subject (Step 502). This may include using subject set-up sub-module 202a of the setup module 202. The information may be stored in subject profile database 242 or other database. Calibration of the subject may also be performed, either manually, automatically and/or semi-automatically (step 504). During the run phase, stimuli and/or survey questions may be presented for display to the subject (Step 506). The subject may answer survey questions manually or otherwise (Step 508). Visual attention data and emotional response data may be collected as described elsewhere herein. In other testing environments, various ones of these steps may be performed without a leader (steps 512-514, 516 and 518).

After the stimuli presentation of the study/survey is completed, it is determined whether another participating subject is available (Step 510, 520). If so, the process may be repeated with another subject. If not, the study session may be concluded and/or analysis may be performed (Step 550).

Analysis may be performed at the conclusion of a test/study and/or in real-time as data is collected. The analysis may include processing the collected data to determine visual attention information and/or emotional response information, among other things. Some aspects of visual attention processing and/or emotional response processing, in general are known. Other aspects are described elsewhere herein.

Eye-tracking, emotional response (and other) calibration techniques, in general are known. Examples of some aspects of the calibration routines that may be used with the invention are provided. Other calibration techniques may also be used. Calibration sub-module 202c performs calibration activity, including subject/device calibration. Eye tracking device 120 and other input devices may be calibrated based on environmental settings and scenarios. Also during calibration, the calibration sub-module 202c may present the subject with a number of calibrations points located in predetermined locations of the display device or the subject's field of vision for subject specific calibration. The calibration points may correspond to coordinates of the display device on which the subject may be prompted to focus and move between until the eye tracking device has calibrated the movement of the subject's eyes in relation to the display device coordinates (e.g., x, y, z coordinates) Optionally, the point calibration information is recorded and stored with the subject profile data for future testing sessions.

Emotional calibration may also be recorded and stored. The subject may be presented with predetermined stimuli used to evoke a certain emotion in order to observe the subjects emotional reaction in relation to their eye properties. A subject may be presented with a stimuli known to elicit a positive (e.g., pleasant), neutral, or negative (e.g., unpleasant) response. By way of example, a subject may be presented with an emotionally neutral stimulus in order to record blink rate pattern, pupil response, saccadic movements, and/or other properties to characterize the subject's response to neutral stimuli. Alternatively, the subject may be presented with stimuli known to evoke a certain emotion based on the subject's demographic and other personal data. The emotional reaction may be used to set an emotional baseline for various emotions. Thus, study/survey stimuli may be compared with a subject's baseline to understand the magnitude of emotional valence.

In connection with running a test, various data may be collected. The collected data may be processed in real-time or subsequently. Collected and processed data may be presented as output information to present visual attention, emotional response and/or other information in a variety of formats as discussed in reference to output presentation properties. One type of output may be a visual output to a display, a visual printout or other visual output. Non-visual output may also be provided.

The output may include a graphical representation including visual attention information (e.g., one or more gaze plot) and/or emotional response information (e.g. one or more emotion meter) for one or more stimuli. The gaze plot(s) (e.g., spotlight(s), attention points, interest points) may be superimposed on the relevant stimulus (or stimuli if two or more are simultaneously displayed). The gaze plot may include a spotlight feature to highlight aggregated fixation points, attention points to highlight temporal ordering of aggregated fixation points and interest points to highlight emotional response.

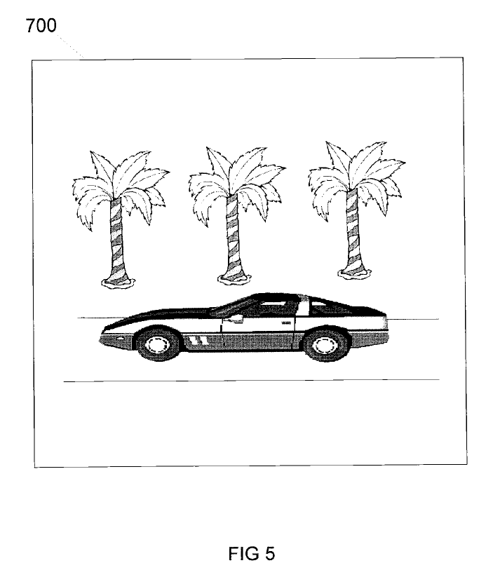

FIG. 5 is an illustration of exemplary visual stimuli, according to an embodiment of the invention. FIG. 6 is an example of an output, according to one aspect of the invention, relating to the stimuli of FIG. 5. The output, as shown, includes a simultaneous display of visual attention information 800 and emotional response information 810 (e.g., an emotion meter) relating to a subject's response to a visual stimulus (e.g., the stimulus 700 of FIG. 5). As shown, the visual attention information 800 includes a gaze plot with a spotlight feature and attention points. The spotlight feature highlights (or otherwise illustrates) one or more fixation points and/or interest points of the visual stimulus 700. In one implementation of the spotlight feature, a virtual mask may be superimposed over all or some of the stimulus (e.g., visual image 700) and portions of the mask, corresponding to one or more fixation points (e.g., based on minimum time of fixation), may be effectively removed or made more transparent to reveal the underlying portion of the stimulus. Another approach is to start with the entire stimulus revealed and selectively mask non-fixation points.

In general, the mask (if used) may have a first set of optical characteristics and the removed portions may have a second set of optical characteristics (e.g., to distinguish the one or more fixation points from the rest of the stimulus). According to one embodiment, the mask may be at least relatively opaque (to fully or partially obscure the underlying portion of the stimulus) and the removed portions corresponding to the fixation points may be made at least relatively more transparent to highlight (or spotlight) the fixation points (as shown for example by pointers 801-804). Areas illustrated by 801, 802, 803, and 804 may also be include attention points for numbering according to the temporal ordering of fixation. If desired, the actual stimulus may be displayed in proximity to the gaze plot to easily see the masked portions of the stimulus.

According to another embodiment, the fixation points may be displayed more brightly than the other points. Other techniques for visually displaying distinctions between fixation points and non-fixation points may be used.

A relative difference in optical characteristics may also be used to indicate the magnitude of fixation points. For example, if a subject dwells at a first fixation point for a longer time than a second fixation point, the first fixation point may be relatively more transparent than the second fixation point, yet each may be more transparent than non-fixation points. Other optical characteristics can be used to distinguish among fixation points and to distinguish fixation points from non-fixation points.

To the extent a user fixates on different points or areas in a particular temporal order, the order of the fixation points may be visually indicated using attention points, either statically or dynamically. If static, the fixation points may be marked with numbers (or other indictors) to match the temporal ordering of one or more fixation points. If dynamic, a first fixation point may be highlighted as compared with other fixation points (e.g., displayed more transparently or more brightly). Then a second and other fixation points may be highlighted in a sequential fashion.

According to another aspect of the invention, a fixation point that is determined to correspond to an emotional response may be referred to as an interest point. One or more interest points may be displayed differently than fixation points that are not associated with an emotional response. Additionally, one interest point may be displayed differently than another interest point based on the determined emotional valence and/or arousal associated with the point or other differences. For example, a spotlight feature may be used to highlight one or more portions/areas of the visual stimulus that correspond to interest points. Characteristics of the interest point spotlights may vary to indicate the type and/or strength of a subject's emotional response associated with the fixation point.

Emotional response information 810 may be displayed simultaneously with visual attention information 800. The emotional response information 810 may include an overall emotional response based on the subject's response to the stimulus (or stimuli) and/or area related emotional response information corresponding to portions of one or more stimuli. For example, a more detailed level of emotional response may be provided by separately displaying emotional response information for one or more fixation points. As shown in FIG. 6, by way of example only, an emotional response meter may show the emotional valence and/or arousal for one or more fixation points. Emotional valence may also be displayed for interest points, spotlights, and/or attention points.

Textual information may be included at various locations on the report, if desired.

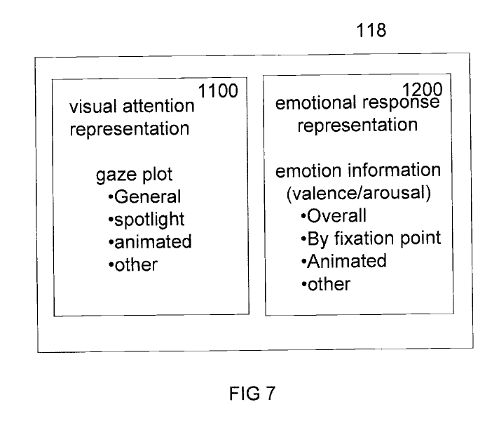

FIG. 7 illustrates some options for display of visual attention information and emotional response information. Various permutations of these features may be used together. Not all features need be used in all cases.

For example, the visual attention information may include a gaze plot (with or without the spotlight feature, attention points, interest points). A gaze plot, if used, may illustrate a scan path corresponding to the subject's eye movements, fixation points, and/or interest points. The visual attention information may be for one or more stimuli at a time. The visual attention information may be static or dynamic. A dynamic display may include a sequence of individual displays (e.g., a slide show mode), animated playback, one or more videos and/or other dynamic displays.

Some output (e.g., reports) may be automatically generated according to one or more templates. Various templates and/or template parameters may be pre-stored in the system. Pre-stored templates can be selected and/or modified (e.g., by an administrative user, test-study leader or other entity). New templates may also be created and stored.

Reports and other output 118 may be automatically sent to one or more recipients and/or recipient devices. For example, subject 50, third party device 250, a study/survey leader, an administrator, and/or other recipient. Output 118 may be stored for later retrieval, transmission, and/or data warehousing. Output and reports can be in any of a number of formats, including without limitation, JPEG, Word, PDF, XML and any other convenient output format.

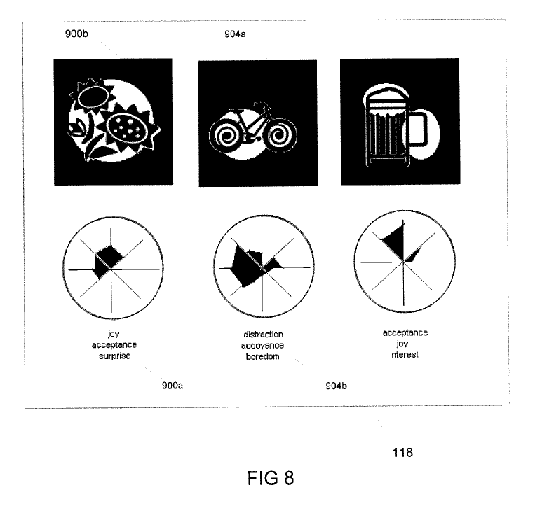

According to an aspect of the invention, emotion maps may be displayed simultaneously and in synchronization with the stimuli that provoked them. For example, as illustrated in FIG. 8, a first gaze plot with spotlight feature for a first stimulus 900a may be displayed in proximity to corresponding emotion map 900b which depicts the emotional response of a subject to stimulus 900a. Similarly, a second gaze plot with spotlight feature for a second stimulus 904a may be displayed in proximity to corresponding emotion map 904b which depicts the emotional response of a subject to stimulus 904a, and so on. Different display formats may be utilized.

Report information along with data from databases (240-243) may be further analyzed for data mining purposes. Data within theses databases, among others, may be used to uncover patterns and relationships contained within the collected data, subject data, and/or analysis results. Background variables (e.g., collected during set-up or other time) including age, gender, location, among others, may be used for data mining. In one or more databases, data mining can be done manually or automatically via data mining module 208 over all or portions of the data.

By way of further explanation, additional information and examples regarding various aspects of the invention is now presented. Survey questions, if used, may be presented one at a time, or a number of survey questions may be shown at one time on a single screen. The order, timing, and display attributes of stimuli may be determined by the administrator and/or subject/survey leader at setup, based on what the administrator may want to analyze. By further example, the administrator may want to study the subject's response to two or more competing market brands. A simultaneous, side by side presentation of stimuli may elicit different visual attention information and emotional reaction with respect to the two or more brands than a sequential display. Other comparative studies may be conducted.

As the study/survey is run, the subject's eye properties and other properties observed by the eye tracking device and/or other sensors may be collected, stored, and/or analyzed. The collected data may be synchronized to a timer for later analysis and/or play back. Collected data may comprise eye property data, other physiological data, environmental data, and/or other data. Collected eye property data may include data relating to a subject's pupil size, blink properties, eye position (or gaze) properties, or other eye properties. Collected pupil data may comprise pupil size, velocity of change (contraction or dilation), acceleration (which may be derived from velocity), or other pupil data. Collected blink data may include, for example, blink frequency, blink duration, blink potention, blink magnitude, or other blink data. Collected gaze data may comprise, for example, saccades, express saccades, nystagmus, or other gaze data. Data relating to the movement of facial muscles (or facial expressions in general) may also be collected. If a subject is presented with stimuli, collected data may be synchronized with the presented stimuli.

Visual attention information components may be decoded from the visual cues (e.g., collected eye property data). This may be done, for example, by applying one or more rules from a visual attention analysis sub-module 205a. Determination and analysis of visual attention may involve various aspects including interest points and interest tracking. Interest points may be based on fixation (gaze) rate and the type of saccades on a portion or portions of the visual stimuli coupled with emotional response as determined by the eye properties. Processing gaze (or eye movement data) may comprise, for example, analyzing saccades, express saccades (e.g., saccades with a velocity greater than approximately 100 degrees per second), and nystagmus (rapid involuntary movements of the eye), or other data. Features of interest may include the velocity (deg/s) and direction of eye movements, fixation time (e.g., how long does the eye focus on one point), the location of the fixation in space (e.g., area as defined by x, y, z or other coordinates), or other features including return to fixation areas, relevance, vergence for depth evaluation, and scan activity.

Visual attention may be determined by setting an adjustable fixation/gazing threshold. A sliding window measured in milliseconds (or other unit of time) can be set as a threshold, for example, 400 ms, in order to determine which points or areas on the visual stimuli the subject gazed at for at least 400 ms. If the user remains fixated on the area for at least the window of time, the area of the visual stimuli may be identified as a fixation point.

The emotional response (e.g., arousal, valence, if any) corresponding to the fixation point may determine the level of interest in that fixation point. For example, if a determined fixation point also elicited an emotional response that exceeds a predetermined emotional threshold value, then the fixation point may be identified as an interest point. Thus, interest points/areas may be identified by the area(s) of a visual stimulus which the subject gazes or fixates upon for more than a predetermined period of time (the selectable threshold value) and elicits a measurable emotional response (emotional threshold value).

If the sliding window threshold is made smaller, for example 100 ms, the subject's entire scan path on the visual stimuli may be revealed. This may allow an administrator or analyzer to see if a specific feature of a visual stimulus was even looked at and for how long.

Graphical representation of the subject's visual attention may be put in the form of a gaze plot.

Emotional response components may include, for example, emotional valence, emotional arousal, emotional category, and/or emotional type. Other components may be determined. Emotional valence may indicate whether a subject's emotional response to a given stimulus is a positive emotional response (e.g., pleasant or “like”), negative emotional response (e.g., unpleasant or “dislike”), or neutral emotional response. Emotional arousal may comprise an indication of the intensity or emotional strength of a response subject a predetermined scale based on the calibrated emotional baseline. Known relationships exist between a subject's emotional valence and arousal and physical properties such as pupil size, blink properties, facial expressions, and eye movement.

Pupil size can range from approximately 1.5 mm to more than 9 mm. Processing pupil data may further comprise determining the velocity of change or how fast a dilation or contraction occurs in response to a stimulus, as well as acceleration which can be derived from velocity. Other pupil-related data including pupil base level and base distance may be determined as well as, for instance, minimum and maximum pupil sizes.

Processing blink data may comprise, for example, determining blink frequency, blink duration, blink potention, blink magnitude, or other blink data. Blink frequency measurement may include determining the timeframe between sudden blink activity.

Blink duration (in, for example, milliseconds) may also be processed to differentiate attentional blinks from physiological blinks. File Blink patterns may be differentiated based on their duration. Neutral blinks may be classified as those which correspond to the blinks measured during calibration. Long blink intervals may indicate increased attention, while short blinks may indicate that the subject may be searching for information. Very short blink intervals may indicate confusion, while half-blinks may serve as an indication of a heightened sense of alert. Blink velocity refers to how fast the amount of eyeball visibility is changing while the magnitude of a blink refers to how much of the eyeball is visible while blinking.

According to another aspect of the invention, analysis module 205 may decode emotional cues from extracted feature data by applying one or more rules from an emotional reaction analysis sub-module 205b to the collected data to determine one or more emotional components.

Business Models

A variety of different business models may be used to exploit the features and advantages of the invention. For example, a service provider may use the software/system to run test centers that subjects physically visit. Tests/studies may be performed on behalf of a third party (e.g. a consumer products company). In this scenario, one or more test leaders may be used to assist/guide the subjects in conjunction with the testing. Self-operated test centers (e.g., kiosks) may also be used with or without a leader. The service provider may collect fees from the third party on a variety of bases. By way of example, the fees may include, but are not limited to, a per test fee per subject, a per test fee for a number of subjects, a per stimuli fee, per segment of subjects and/or other bases. Additionally, the amount of fee may vary depending on the type/detail of output. For example, a simple visual attention output (e.g., gaze plot only) may be provided for a first fee. More detailed information (e.g., a gaze plot with the spotlight feature) may be a second fee. A simultaneous display of visual attention information (e.g., a gaze plot with or without a spotlight feature) along with basic emotional response information may be a third fee. Adding more detailed emotional response information (e.g., emotional response for one or more fixation points) may be a fourth fee. Other types of outputs, video, animated, etc. may command other fees. Other business models for such service providers may be implemented.

According to another business method, a service provider may operate a remotely accessible (via the Internet or other network) test facility with which subjects can interact remotely. The subject can access the remotely accessible test facility in any of a number of ways, including but not limited to, via a test center, a kiosk, a home or work computer, a mobile wireless device or otherwise. Fees may be charged as indicated above or otherwise.

According to another aspect of the invention, an invoice module (e.g., invoice module 207) may be used to at least partially automate the process of billing. The invoice module 207 may monitor system information and automatically determine fees and generate invoices. Fee information may be input during a setup phase or otherwise. The monitored information may include test run, subject tested, stimuli presented, type and/or level of detail of output and/or other information upon which fees maybe based.

In the foregoing specification, the invention has been described with reference to specific embodiments thereof. Various modifications and changes may be made thereto without departing from the broader spirit and scope of the invention. The specification and drawings are, accordingly, to be regarded in an illustrative rather than a restrictive sense.

1. A computer-implemented method for detecting a subject's emotional response to one or more stimuli, comprising:
presenting at least one stimulus to a subject;
collecting physiological data from the subject while the at least one stimulus is presented to the subject to enable determination of an emotional response;
processing the physiological data, via a processor, to determine visual attention information;
generating emotional response information in response to the at least one stimulus; and
generating, for the at least one stimulus, a representation of the determined visual attention information, and a representation of the generated emotional response information.

2. The method of claim 1, wherein the at least one stimulus is a visual stimulus.

3. The method of claim 2, wherein the visual stimulus comprises at least one of text, a picture, artwork, a movie, a multimedia presentation, or interactive content.

4. The method of claim 2, wherein the visual stimulus comprises at least one of an advertisement, or a commercial.

5. The method of claim 2, wherein the visual stimulus comprises a depiction of a product.

6. The method of claim 2, wherein the visual stimulus comprises product packaging.

7. The method of claim 1, wherein the at least one stimulus comprises a visual stimulus and at least one non-visual stimulus.

8. The method of claim 1, wherein the physiological data includes one or more of pupil data, blink data, or gaze data.

9. The method of claim 1, wherein the physiological data is collected via an eye-tracking device.

10. The method of claim 1, wherein the representation of the determined visual attention information and the representation of the determined emotional response information are output to a display or printer.

11. The method of claim 1, wherein determining the visual attention information comprises:
determining one or more fixation points associated with the at least one stimulus, wherein a fixation point comprises an area on which the subject visually focused for at least a predetermined amount of time.

12. The method of claim 11, wherein the representation of the determined visual attention information comprises a gaze plot superimposed on the at least one stimulus presented to the subject, and wherein the one or more fixation points are highlighted so as to distinguish the one or more fixation points from the remainder of the at least one stimulus.

13. The method of claim 12, wherein the representation of the generated emotional response information includes emotional information corresponding to each of the one or more highlighted fixation points.

14. The method of claim 11, further comprising:
aggregating the one or more fixation points so as to identify one or more attention points.

15. The method of claim 14, further comprising:
aggregating the one or more fixation points with temporal ordering so as to identify one or more attention points.

16. The method of claim 15, further comprising:
numbering the one or more identified attention points to indicate the temporal ordering.

17. The method of claim 11, furthering comprising:
determining if the emotional response information corresponds to any of the one or more fixation points so as to identify one or more interest points.

18. The method of claim 17, wherein the representation of the generated emotional response information includes an indication of emotional valence and emotional arousal for each identified interest point.

19. The method of claim 1, wherein emotional response information includes at least one of an emotional valence component or an emotional arousal component.

20. The method of claim 1, wherein the representation of the generated emotional response information includes emotional information indicative of an overall emotional response to the at least one stimulus.

21. The method of claim 1, further comprising:
prompting the subject to answer one or more survey questions corresponding to the at least one stimulus presented to the subject.

22. The method of claim 1, wherein presenting the at least one stimulus to the subject and collecting the physiological data from the subject occurs at a kiosk; and wherein processing the physiological data to determine visual attention information, and generating emotional response information occurs at a processing center located remotely from the kiosk.

23. The method of claim 1, wherein the at least one stimulus comprises an advertisement provided by an entity, and wherein the representation of the determined visual attention information and the representation of the generated emotional response information are provided to the entity for evaluation.

24. The method of claim 1, wherein the at least one stimulus is presented to a plurality of subjects, and wherein the representation of the determined visual attention information and the representation of the generated emotional response information each depict an aggregate of the visual attention information and emotional response information determined for the plurality of subjects.

25. A computer-implemented system for detecting a subject's emotional response to one or more stimuli, comprising:
means for presenting at least one stimulus to a subject;
means for collecting physiological data from the subject while the at least one stimulus is presented to the subject to enable determination of an emotional response;
means for processing the physiological data to determine visual attention information;
means for generating emotional response information in response to the at least one stimulus; and
means for generating, for the at least one stimulus, a representation of the determined visual attention information, and a representation of the generated emotional response information.

26. The system of claim 25, wherein the at least one stimulus is a visual stimulus.

27. The system of claim 26, wherein the visual stimulus comprises at least one of text, a picture, artwork, a movie, a multimedia presentation, or interactive content.

28. The system of claim 26, wherein the visual stimulus comprises at least one of an advertisement, or a commercial.

29. The system of claim 26, wherein the visual stimulus comprises a depiction of a product.

30. The system of claim 26, wherein the visual stimulus comprises product packaging.

31. The system of claim 25, wherein the at least one stimulus comprises a visual stimulus and at least one non-visual stimulus.

32. The system of claim 25, wherein the physiological data includes one or more of pupil data, blink data, or gaze data.

33. The system of claim 25, wherein the physiological data is collected via an eye-tracking device.

34. The system of claim 25, wherein the representation of the determined visual attention information and the representation of the determined emotional response information are output to a display or printer.

35. The system of claim 25, wherein the means for processing the physiological data to determine visual attention information further comprises means for determining one or more fixation points associated with the at least one stimulus, wherein a fixation point comprises an area on which the subject visually focused for at least a predetermined amount of time.

36. The system of claim 35, wherein the representation of the determined visual attention information comprises a gaze plot superimposed on the at least one stimulus presented to the subject, and wherein the one or more fixation points are highlighted so as to distinguish the one or more fixation points from the remainder of the at least one stimulus.

37. The system of claim 36, wherein the representation of the generated emotional response information includes emotional information corresponding to each of the one or more highlighted fixation points.

38. The system of claim 35, further comprising:
means for aggregating the one or more fixation points so as to identify one or more attention points.

39. The system of claim 38, further comprising:
means for aggregating the one or more fixation points with temporal ordering so as to identify one or more attention points.

40. The system of claim 39, further comprising:
means for numbering the one or more identified attention points to indicate the temporal ordering.

41. The system of claim 35, furthering comprising:
means for determining if the emotional response information corresponds to any of the one or more fixation points so as to identify one or more interest points.

42. The system of claim 41, wherein the representation of the generated emotional response information includes an indication of emotional valence and emotional arousal for each identified interest point.

43. The system of claim 25, wherein emotional response information includes at least one of an emotional valence component or an emotional arousal component.

44. The system of claim 25, wherein the representation of the generated emotional response information includes emotional information indicative of an overall emotional response to the at least one stimulus.

45. The system of claim 25, further comprising:
means for prompting the subject to answer one or more survey questions corresponding to the at least one stimulus presented to the subject.

46. The system of claim 25, wherein the means for presenting the at least one stimulus to the subject and the means for collecting the physiological data from the subject are located at a kiosk; and wherein means for processing the physiological data to determine visual attention information, and the means for generating emotional response information are located at a processing center remote from the kiosk.

47. The system of claim 25, wherein the at least one stimulus comprises an advertisement provided by an entity, and wherein the representation of the determined visual attention information and the representation of the generated emotional response information are provided to the entity for evaluation.

48. The system of claim 25, wherein the at least one stimulus is presented to a plurality of subjects, and wherein the representation of the determined visual attention information and the representation of the generated emotional response information each depict an aggregate of the visual attention information and emotional response information determined for the plurality of subjects.
